(19) United States
(12) Patent Application Publication
(10) Pub. No.: US 2024/0394387 A1
(43) Pub. Date: Nov. 28, 2024

(54) ELECTRONIC DEVICE AND METHOD OF ENCRYPTING BIO-SIGNALS USING SAME

(71) Applicants: SAMSUNG ELECTRONICS CO., LTD., Suwon-si (KR); SEOUL NATIONAL UNIVERSITY R&DB FOUNDATION, Seoul (KR)

(72) Inventors: Youngsoo Kim, Suwon-si (KR); Jong Ae Park, Yongin-si (KR)

(73) Assignees: SAMSUNG ELECTRONICS CO., LTD., Suwon-si (KR); SEOUL NATIONAL UNIVERSITY R&DB FOUNDATION, Seoul (KR)

(21) Appl. No.: 18/370,214

(22) Filed: Sep. 19, 2023

(30) Foreign Application Priority Data
May 25, 2023 (KR) 10-2023-0067635

(51) Int. Cl. G06F 21/60; A61B 5/00

(52) U.S. Cl. CPC G06F 21/606; A61B 5/0004; A61B 5/742; G06F 21/602

(57) ABSTRACT

An electronic device may include: a sensor configured to measure a plurality of bio-signals that includes a first bio-signal and one or more second bio-signals; an encryption chip configured to encrypt the first bio-signal based on a first encryption method that uses the one or more second bio-signals as an encryption code, and output the encrypted first bio-signal; and a communication device configured to transmit the encrypted first bio-signal to an external device.

### CROSS-REFERENCE TO RELATED APPLICATION(S)

This application claims priority from Korean Patent Application No. 10-2023-0067635, filed on May 25, 2023, in the Korean Intellectual Property Office, the disclosure of which is incorporated herein by reference in its entirety.

### BACKGROUND

### 1. Field

Apparatuses and methods consistent with example embodiments relate to encrypting personal bio-signals.

### 2. Description of the Related Art

A method of using personal bio-signals is largely divided into two methods. In one method, each individual's unique characteristics, including fingerprint, iris, face, vein recognition, etc., are used as a means of authentication, and in the other method, bio-signals, such as electrocardiogram, blood pressure, blood glucose, skin, etc., are used to provide personal health information to individuals.

The former method is used for authentication purposes, such that a security-related authentication process has been developed along with improved security technology. However, a recent technology using bio-signals, such as heart rate, electrocardiogram, blood pressure, blood glucose, etc., is started to be used in mobile devices and wearable devices and is in its infancy with relatively poor performance.

Up until now, the bio-signals have been mainly used within hospital settings, limiting direct access to such information by individuals or third parties. However, a recent development of technology applied to mobile devices and wearable devices allows the third party, including individuals themselves, to easily access personal bio-signals, thereby increasing the possibility of misuse of personal bio-signals by others. In addition, for companies using the bio-signals in mobile terminals and wearable devices, misuse of bio-information may be an obstacle to commercialization compared to the benefits of using the bio-signals. For this reason, there is a desperate need for solutions to security issues of personal bio-signals.

### SUMMARY

According to an aspect of the present disclosure, an electronic device may include: a sensor configured to measure a plurality of bio-signals that includes a first bio-signal and one or more second bio-signals: an encryption chip configured to encrypt the first bio-signal based on a first encryption method that uses the one or more second bio-signals as an encryption code, and output the encrypted first bio-signal; and a communication device configured to transmit the encrypted first bio-signal to an external device.

The first encryption method may be performed by applying a constant to the one or more second bio-signals to obtain weighted one or more second bio-signals, and by combining the weighted one or more second bio-signals with the first bio-signal.

The constant may be a predetermined time delay constant.

The encryption chip may be configured to encrypt the one or more second bio-signals by a second encryption method. The communication device may be configured to transmit the encrypted one or more second bio-signals along with the first bio-signal.

The plurality of bio-signals may be signals that change continuously over time, and includes at least one of an electrocardiogram signal, blood pressure, blood glucose, heart rate, oxygen saturation (SpO₂), arterial stiffness, stress index, body fat, and body temperature.

The electronic device may further include a display configured to display the plurality of bio-signals before encryption is applied to the plurality of bio-signals.

The display may include a user interface configured to receive a user input that indicates the first bio-signal as an encryption target.

The electronic device may further include a decryption chip configured to receive an encrypted third bio-signal and unencrypted one or more fourth bio-signals, decrypt the encrypted third bio-signal by using the unencrypted one or more fourth bio-signals as a decryption code, and the decrypted third bio-signal.

The encryption chip may be configured to generate a decryption key based on the one or more second bio-signals, and the communication device is further configured to share the decryption key with the external device.

The first bio-signal and the one or more second bio-signals may be different types of bio-signals which are simultaneously measured by the sensor.

According to another aspect of the present disclosure, a bio-signal encryption chip may include: a receiver configured to receive a first bio-signal and one or more second bio-signals: a converter configured to convert the first bio-signal and the one or more second bio-signals into digital signals; and a processor configured to encrypt the converted first bio-signal based on a predetermined encryption method that uses the converted one or more second bio-signals as an encryption code, and output the encrypted first bio-signal.

The predetermined encryption method may be performed by applying a constant to the converted one or more second bio-signals to obtain weighted one or more second bio-signals, and by combining the weighted one or more second bio-signals with the converted first bio-signal.

The constant may be a predetermined time delay constant.

According to another aspect of the present disclosure, a method of encrypting bio-signals by an electronic device, may include: measuring a plurality of bio-signals that includes a first bio-signal and one or more second bio-signals: encrypting the first bio-signal based on a first encryption method that uses the one or more second bio-signals as an encryption code; and transmitting the encrypted first bio-signal to an external device.

The first encryption method is performed by applying a constant to the one or more second bio-signals to obtain weighted one or more second bio-signals and by combining the weighted one or more second bio-signals with the first bio-signal.

The constant is a predetermined time delay constant.

The method may further include encrypting the one or more second bio-signals by a second encryption method, and transmitting the encrypted one or more second bio-signals along with the first bio-signal to the external device.

The method may further include displaying the plurality of bio-signals before encryption is applied to the plurality of bio-signals.

The method may further include: generating a decryption key based on the one or more second bio-signals; and sharing the decryption key with the external device.

### BRIEF DESCRIPTION OF THE DRAWINGS

FIGS. 1A and 1B are block diagrams illustrating an electronic device according to an embodiment of the present disclosure.

FIG. 2 is a block diagram illustrating an encryption chip in an electronic device according to an embodiment of the present disclosure.

FIGS. 3A, 3B, and 3C are graphs showing a process of encrypting a bio-signal according to an embodiment of the present disclosure.

FIG. 4 is a diagram illustrating a plurality of bio-signals displayed on a display.

FIG. 5 is a flowchart illustrating a method of encrypting bio-signals according to an embodiment of the present disclosure.

FIGS. 6, 7, 8, and 9 are diagrams illustrating examples of structures of an electronic device.

### DETAILED DESCRIPTION

Example embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the example embodiments. However, it is apparent that the example embodiments can be practiced without those specifically defined matters. Also, well-known functions or constructions are not described in detail since they would obscure the description with unnecessary detail.

It will be understood that, although the terms first, second, etc. may be used herein to describe various elements, these elements should not be limited by these terms. These terms are only used to distinguish one element from another. Any references to singular may include plural unless expressly stated otherwise. In addition, unless explicitly described to the contrary, an expression such as “comprising” or “including” will be understood to imply the inclusion of stated elements but not the exclusion of any other elements. Also, the terms, such as “unit” or “module,” etc., should be understood as a unit that performs at least one function or operation and that may be embodied as hardware, software, or a combination thereof.

In addition, the expression, such as “at least one of, for example, at least one of a, b, and c,” can be construed as including only a, only b, or only c, a and b, a and c, b and c, or a, b, and c.

An electronic device according to various embodiments of the present disclosure which will be described below may include, for example, at least one of a wearable device, a smartphone, a tablet PC, a mobile phone, a video phone, an electronic book reader, a desktop computer, a laptop computer, a netbook computer, a workstation, a server, a PDA, a portable multimedia player (PMP), an MP3 player, a medical device, and a camera. The wearable device may include at least one of an accessory type wearable device (e.g., wristwatch, ring, bracelet, anklet, necklace, glasses, contact lens, or head mounted device (HMD)), a textile/clothing type wearable device (e.g., electronic clothing), a body-mounted type wearable device (e.g., skin pad or tattoo), and a body implantable type wearable device. However, the wearable device is not limited thereto and may include, for example, various types of medical equipment including various portable medical measuring devices (antioxidant measuring device, blood glucose monitor, heart rate monitor, blood pressure measuring device, thermometer, etc.), magnetic resonance angiography (MRA), magnetic resonance imaging (MRI), computed tomography (CT), imaging system, ultrasonic system, etc.) and the like. However, the electronic device is not limited to the above devices.

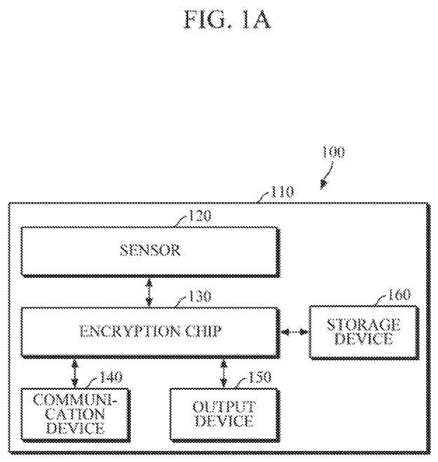

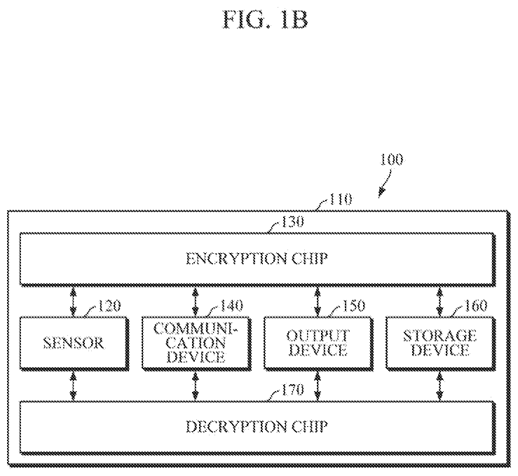

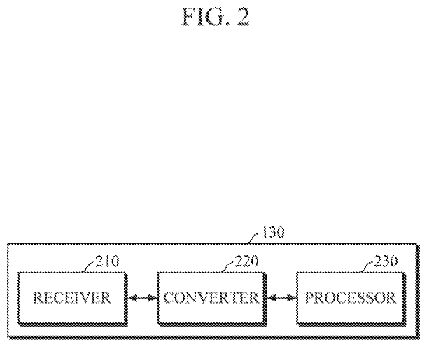

FIGS. 1A and 1B are block diagrams illustrating an electronic device according to an embodiment of the present disclosure. FIG. 2 is a block diagram illustrating an encryption chip in an electronic device according to an embodiment of the present disclosure.

Referring to FIG. 1A, an electronic device 100 includes a sensor 120, an encryption chip 130, a communication device (e.g., a communication interface) 140, an output device 150, and a storage device 160 which are mounted in a main body 110.

The sensor 120, provided in the main body 110, may measure a plurality of bio-signals. In this case, the plurality of bio-signals may be signals that are measurable by, for example, a Photoplethysmography (PPG) sensor or an Electrocardiogram (ECG) sensor, etc., and that change continuously over time, and may include at least one of electrocardiogram, blood pressure, blood glucose, heart rate, oxygen saturation (SpO₂), arterial stiffness, stress index, body fat, and body temperature. However, the types of bio-signals and sensors for measuring the bio-signals are not limited thereto.

The encryption chip 130 may receive the plurality of bio-signals measured by the sensor 120, and may encrypt a first bio-signal selected from the plurality of bio-signals for transmission to an external device, by using one or more second bio-signals selected from the plurality of bio-signals. For example, the encryption chip 130 may encrypt the first bio-signal based on a first encryption method in which the one or more second bio-signals are used as an encryption code, and output the encrypted first bio-signal to the external device. The plurality of bio-signals may be measured by the sensor 120 at the same time.

In particular, the first encryption method may be a method of combining the first bio-signal with the one or more second bio-signals, for example, by applying a predetermined constant to the one or more second bio-signals and combining an application result with the first bio-signal. The predetermined constant may be a time delay constant (e.g., one second), but is not limited thereto.

The following Equation 1 represents a method of generating the encrypted first bio-signal.

$$Z = X + α Y \qquad [ Equation 1 ]$$

Herein, Z denotes the encrypted first bio-signal, X denotes the first bio-signal before encryption, Y denotes the second bio-signal, and a denotes the time delay constant. In Equation 1, the first bio-signal and the second bio-signal are combined by addition, but the combination is not limited thereto, and various combination methods may also be used including subtraction, multiplication, division and the like of the first bio-signal and the second bio-signal. In addition, the encrypted first bio-signal may also be generated by applying a predetermined function based on circuit characteristics of the encryption chip to the second bio-signal and by combining a value, obtained by multiplying an application result by the time delay constant, with the first bio-signal. Further, while only one second bio-signal is used in Equation 1, a plurality of second bio-signals may be used, and a plurality of predetermined constants to be combined with the second bio-signals may also be used.

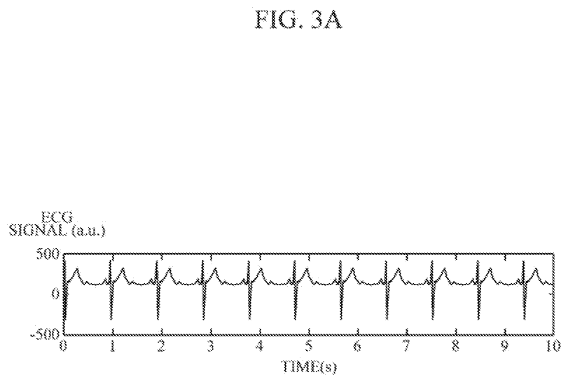

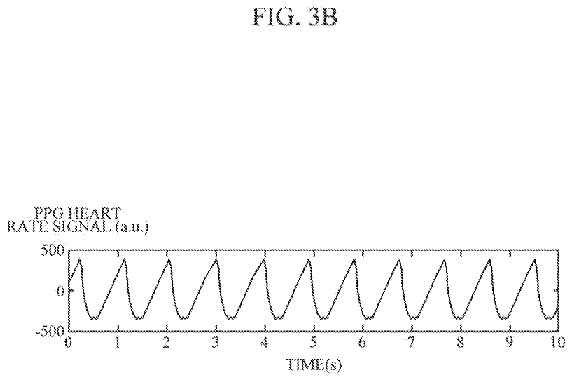

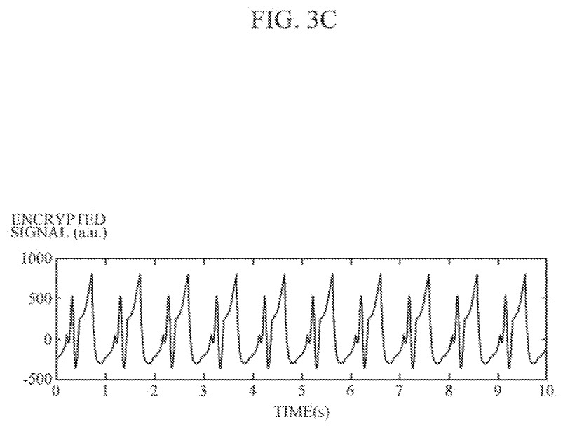

FIGS. 3A to 3C are graphs showing a process of encrypting a first bio-signal according to an embodiment of the present disclosure.

FIG. 3A is a graph over time of a first bio-signal (e.g. ECG signal) selected to be encrypted, FIG. 3B is a graph over time of a second bio-signal (e.g., PPG signal indicating heart rate) used as an encryption code, and FIG. 3C is a graph over time of an encrypted first bio-signal.

If the time delay constant is one second in the above Equation 1, the bio-signal shown in the graph of FIG. 3C is a bio-signal obtained by applying a time delay of one second to the second bio-signal of FIG. 3B and combining an application result with the first bio-signal of FIG. 3A, and it can be seen that the encrypted bio-signal of FIG. 3C is totally different from the first bio-signal corresponding to the ECG signal and the PPG signal indicating the heart rate.

Referring back to FIG. 2, more specifically, the encryption chip 130 includes a receiver 210, a converter 220, and a processor 230.

The receiver 210 may receive a plurality of bio-signals measured by the sensor 120, and may receive, for example, a first bio-signal to be encrypted and one or more second bio-signals except the first bio-signal, and may include a multiplexer (MUX). The receiver 210 may be implemented as an analog circuit.

The converter 220 may convert the received first and second bio-signals into digital signals, and may include an analog to digital converter (ADC).

The processor 230 may encrypt the first bio-signal, converted into the digital signal, by using as an encryption code one or more second bio-signals converted into the digital signals and using a predetermined encryption method, and may output the encrypted first bio-signal. In this case, as shown in the above Equation 1, the predetermined encryption method may be a method of encrypting the first bio-signal by applying a constant (e.g., predetermined time delay constant) to the one or more second bio-signals converted into the digital signals, and combining an application result with the first bio-signal converted into the digital signal.

The encryption chip 130 may selectively encrypt input signals rather than encrypting all the input signals. For example, the processor 230 of the encryption chip 130 may output a signal processing result of the unencrypted first bio-signal and the unencrypted second bio-signal, along with the encrypted first bio-signal. The electronic device 100 may display the signal processing result of the unencrypted first bio-signal and the unencrypted second bio-signal through the output device 150 (e.g., a display or a speaker) of the electronic device 100, and may transmit the encrypted first bio-signal to the external device through the communication device 140.

Referring back to FIG. 1A, the communication device 140 may be a device for communication with an external device and may transmit the encrypted first bio-signal, output from the encryption chip 130, to the external device. In this case, the external device may be a server for performing big data analysis or artificial intelligence (AI) analysis by using, for example, the bio-signals, and if the bio-signal analysis is performed in a personal terminal, the external device may be another terminal such as a smartphone or a PC. The external device is not limited thereto.

The communication device 140 may share a cryptographic or encryption key and a decryption key corresponding to the cryptographic or encryption key, with the external device before transmitting the encrypted first bio-signal, and may transmit first bio-information only to the external device that shares the encryption key.

The communication device 140 may transmit a bio-signal only to a server or a terminal that is mutually confirmed, and may share unique information based on an encryption chip, e.g., a serial number of an encryption chip, as the encryption key with the server or terminal, so that the bio-signal may be decrypted only in the mutually confirmed server or terminal. As an example, before transmitting an encrypted first bio-signal (which is encrypted using one or more second bio-signals measured simultaneously with the first bio-signal) and a corresponding decryption key generated from the second bio-signals, the electronic device 100 shares the serial number of the encryption chip 130 with an external device. This facilitates a two-step decryption process by the external device, wherein the serial number of the encryption chip 130 and the second bio-signals are transmitted from the electronic device 100 to the external device as decryption keys for decrypting the encrypted first bio-signal to ensure data integrity and confidentiality. In addition, the communication device 140 may share, as the decryption key, a function based on circuit characteristics of the encryption chip which is used in the encryption process. However, the shared decryption key is not limited thereto.

Further, the communication device 140 may transmit the (unencrypted) second bio-signals along with the (encrypted) first bio-signal to the external device. In this case, upon receiving the signals, the external device may decrypt the first bio-signal by a method corresponding to the first encryption method, e.g., by using the second bio-signals as a decryption code, i.e., applying a predetermined constant (e.g., time advance of one second) to the second bio-signal and combining a value, obtained as a result of the application, with the encrypted first bio-signal.

When transmitting the second bio-signals along with the encrypted first bio-signal to the external device, the communication device 140 may also encrypt the second bio-signals by a second encryption method and may transmit the encrypted second bio-signals. In this case, the second encryption method may be another general encryption method different from the first encryption method, and may be a Wireless LAN (WLAN) encryption method, e.g., wired equivalent privacy (WEP), Wi-Fi protected access (WPA), and the like.

The output device 150 may display a plurality of bio-signals before encryption on a display, and may receive, through the display, a first bio-signal determined by user selection among the plurality of bio-signals displayed on the display.

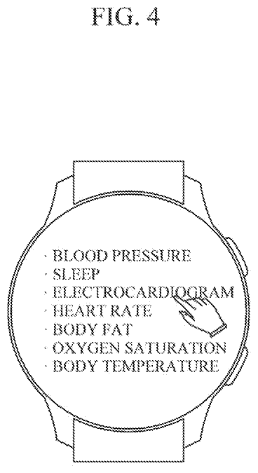

FIG. 4 is a diagram illustrating a plurality of bio-signals before encryption which are displayed on a display.

For example, a user may select a first bio-signal (e.g., electrocardiogram) to be encrypted and transmitted to an external device among the plurality of bio-signals displayed on the display, and the output device 150 may receive the selected first bio-signal through the display. In this case, the encryption chip 130 may determine, based on predetermined criteria or randomly, one or more second bio-signals to be used as an encryption code for the selected first bio-signal. The predetermined criteria may be information, such as heart rate, oxygen saturation, etc., which has low importance compared to main health information, such as blood pressure, blood glucose, electrocardiogram, etc., which is generally desired by a user to receive. Further, the user may select not only the first bio-signal but also a second bio-signal for use as an encryption code directly through the display, and the encryption chip 130 may encrypt the first bio-signal by using the first and second bio-signals selected by the user.

The storage device 160 may store information related to the bio-signal encryption. For example, the storage device 160 may store the time delay constant, encryption key, decryption key, unique information of the encryption chip, and the like.

The storage device 160 may include a storage medium having at least one type of a flash memory type, a hard disk type, a multimedia card micro type, a card type (e.g., a SD memory, a XD memory, etc.), a random access memory (RAM), a static random access memory (SRAM), a read-only memory (ROM), an electrically erasable programmable read-only memory (EEPROM), a programmable read-only memory (PROM), a magnetic memory, a magnetic disk, or an optical disk, etc., but is not limited thereto.

Referring back to FIG. 1B, the communication device 140 may receive a plurality of bio-signals. In this case, the electronic device 100 may further include a decryption chip 170 which, by using as a decryption code one or more fourth bio-signals, other than an encrypted third bio-signal, among the received plurality of bio-signals, is configured to decrypt the encrypted third bio-signal and output the decrypted third bio-signal.

A decryption method by the decryption chip 170 may be a method corresponding to the aforementioned first encryption method and may include, for example, decrypting the third bio-signal by applying a predetermined constant (e.g., time advance of one second) to the fourth bio-signal and by combining a value, obtained as a result of the application, with the encrypted third bio-signal. In this case, the encryption chip 130 and the decryption chip 170 may be formed as one chip so that encryption and decryption may be performed by the one chip.

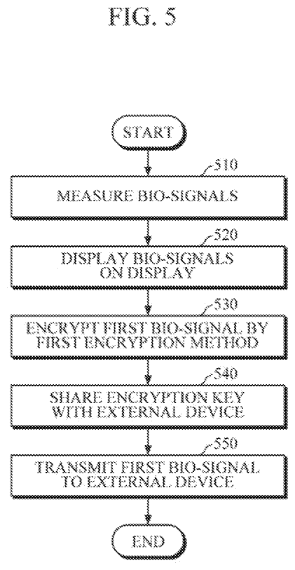

FIG. 5 is a flowchart illustrating a method of encrypting bio-signals according to an embodiment of the present disclosure.

FIG. 5 is an example of a method of encrypting bio-signals by the electronic device 100 according to the embodiments of the FIGS. 1A and 1B, which are described in detail above, and thus will be briefly described below in order to avoid redundancy.

Referring to FIG. 5, the electronic device may first measure a plurality of bio-signals in operation 510, and may display the measured bio-signals on a display in operation 520. For example, the electronic device may receive a user input that selects a first bio-signal as an encryption target, among the plurality of bio-signals displayed on the display. The display may include a user interface configured to receive the user input.

Then, the electronic device may encrypt the first bio-signal based on a first encryption method that uses one or more second bio-signals that are selected from the plurality of bio-signals as an encryption code, and may output the encrypted first bio-signal in operation 530. In this case, the first encryption method may be a method of encrypting the first bio-signal by applying a predetermined constant to the one or more second bio-signals and combining an application result with the first bio-signal, in which the predetermined constant may be a time delay constant.

Then, the electronic device may share a cryptographic or encryption key, and a corresponding decryption key, with an external device in operation 540. Before transmitting the encrypted first bio-signal, the electronic device may transmit a bio-signal only to a server or a terminal that is mutually confirmed, and may share unique information based on an encryption chip, e.g., a serial number of an encryption chip, as the encryption key or the decryption key with the server or terminal, so that the bio-signal may be decrypted only in the mutually confirmed server or terminal.

Subsequently, the electronic device may transmit the encrypted first bio-signal to the external device that shares the encryption key and/or the decryption key in operation 550. In this case, the electronic device may transmit the second bio-signals along with the first bio-signal to the external device. Upon receiving the signals, the external device may decrypt the first bio-signal by a method corresponding to the first encryption method, e.g., by using the second bio-signals as a decryption code, i.e., applying a predetermined constant (e.g., time advance of one second) to the second bio-signals and combining a value, obtained as a result of the application, with the encrypted first bio-signal. In this case, the electronic device may determine the second bio-signals based on predetermined criteria or randomly.

When transmitting the second bio-signals along with the encrypted first bio-signal to the external device, the electronic device may also encrypt the second bio-signals by a second encryption method and may transmit the encrypted second bio-signals. In this case, the second encryption method may be another general encryption method different from the first encryption method, and may be a Wireless LAN (WLAN) encryption method, e.g., wired equivalent privacy (WEP), Wi-Fi protected access (WPA), and the like.

With the development of healthcare technology using wearable devices, security of bio-signals, which are individuals' unique information, is considered as an important issue. According to the above embodiment, bio-information, displayed on the display of a wearable device, and bio-information transmitted to the external device, are encrypted into different information by an encryption process, thereby enhancing the security of personal bio-information. In addition, by encrypting bio-signals directly in the encryption chip, the encryption process may be simplified and less time is required.

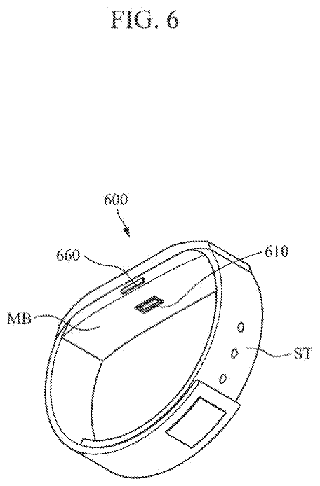

FIGS. 6 to 9 are diagrams illustrating examples of structures of an electronic device.

Referring to FIG. 6, the electronic device may be implemented as a smart watch-type wearable device 600 which includes a main body MB and a wrist strap ST.

The main body MB may be formed in various shapes. A battery may be embedded in the main body MB and/or the strap ST to supply power to various components of the wearable device. The strap ST may be connected to both ends of the main body to allow the main body to be worn on a user's wrist, and may be flexible so as to be wrapped around the user's wrist. The strap ST may be composed of a first strap and a second strap which are separated from each other. One ends of the first and second straps are connected to both sides of the main body MB, and the first and second straps may be connected to each other via a fastening means formed at the other ends of the first and second straps. In this case, the fastening means may be formed as magnetic fastening, Velcro fastening, pin fastening, etc., but is not limited thereto. Further, the strap ST is not limited thereto, and may be integrally formed as a non-detachable band.

The main body MB may include a sensor 610, an encryption chip, a communication device, an output device, a storage device, a decryption chip, and the like. However, depending on the size and shape of a form factor and the like, some of the output device, the storage device, and the decryption chip may be omitted, and the encryption chip and the decryption chip may be formed as one chip so that encryption and decryption may be performed by the one chip.

In this case, the encryption chip may include a receiver configured to receive a first bio-signal and one or more second bio-signals, a converter configured to convert the received first bio-signal and one or more second bio-signals into digital signals, and a processor configured to encrypt the first bio-signal, converted into the digital signal, by using as an encryption code the one or more second bio-signals converted into the digital signals and using a predetermined encryption method, and to output the encrypted first bio-signal.

A manipulator 660 may be formed on a side surface of the main body MB, as illustrated herein. The manipulator 660 may receive a user command and may have a power button to turn on/off the wearable device 600.

A display may be provided on a front surface of the main body MB and may display various application screens, including bio-information, time information, received message information, and the like.

For example, a user may select a first bio-signal (e.g., electrocardiogram) to be encrypted and transmitted to an external device among the plurality of bio-signals displayed on the display, and the output device may receive the selected first bio-signal through the display.

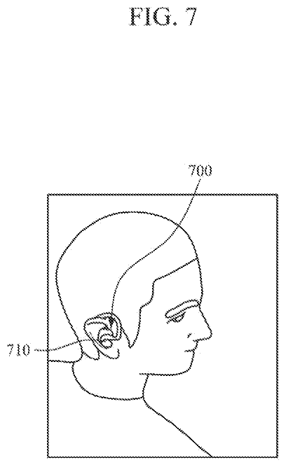

Referring to FIG. 7, the electronic device may also be implemented as an ear-wearable device 700.

The ear-wearable device 700 may include a main body and an ear strap. A user may wear the ear-wearable device 700 by wearing the ear strap on the auricle. The ear strap may be omitted depending on the shape of the ear-wearable device 700. The main body may be inserted into the external auditory meatus. A sensor, an encryption chip, a communication device, an output device, and the like may be provided in the main body. The ear-wearable device 700 may transmit an encrypted bio-signal to an external device through the communication device provided in the main body.

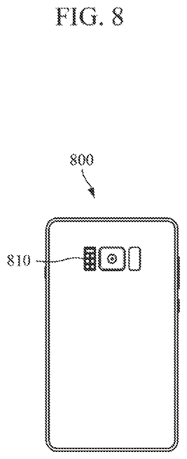

Referring to FIG. 8, the electronic device may be implemented as a mobile device 800 such as a smartphone.

The mobile device 800 may include a housing and a display panel. The housing may form the exterior of the mobile device 800. The housing has a first surface, on which a display panel and a cover glass may be disposed sequentially, and the display panel may be exposed to the outside through the cover glass. A sensor 810, a camera module and/or an infrared sensor, and the like may be disposed on a second surface of the housing.

For example, a plurality of sensors for obtaining data from a user may be disposed on a rear surface of a main body of the mobile device 800, including a fingerprint sensor disposed on a front surface of the mobile device 800, sensors disposed at a power button or a volume button on a side surface thereof or disposed at other positions of the front and rear surfaces of the mobile device 800, and the like.

In addition, in response to a user's request to transmit a bio-signal to an external device by executing an application and the like installed in the mobile device 800, the mobile device 800 may encrypt the bio-signal by using an encryption chip in the mobile device and may transmit the encrypted bio-signal to the external device.

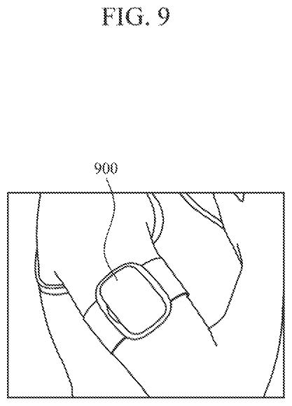

Referring to FIG. 9, an electronic device 900 may be implemented as a patch-type device.

For example, the electronic device 900 may be fixed to a body measurement location (e.g., upper arm, chest, etc.) by a strap, and may acquire a bio-signal of a user. In this case, the electronic device 900 may encrypt the acquired bio-signal by using an encryption chip provided in the electronic device 900, and may transmit the encrypted bio-signal to an external device, e.g., a server of a medical institution, a mobile device, a tablet PC, another medical device, etc., through a communication device provided in the electronic device 900.

While not restricted thereto, an example embodiment can be embodied as computer-readable code on a computer-readable recording medium. The computer-readable recording medium is any data storage device that can store data that can be thereafter read by a computer system. Examples of the computer-readable recording medium include read-only memory (ROM), random-access memory (RAM), CD-ROMs, magnetic tapes, floppy disks, and optical data storage devices. The computer-readable recording medium can also be distributed over network-coupled computer systems so that the computer-readable code is stored and executed in a distributed fashion. Also, an example embodiment may be written as a computer program transmitted over a computer-readable transmission medium, such as a carrier wave, and received and implemented in general-use or special-purpose digital computers that execute the programs. Moreover, it is understood that in example embodiments, one or more units of the above-described apparatuses and devices can include circuitry, a processor, a microprocessor, etc., and may execute a computer program stored in a computer-readable medium.

The foregoing exemplary embodiments are merely exemplary and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. An electronic device comprising:
a sensor configured to measure a plurality of bio-signals that comprises a first bio-signal and one or more second bio-signals;
an encryption chip configured to encrypt the first bio-signal based on a first encryption method that uses the one or more second bio-signals as an encryption code, and output the encrypted first bio-signal; and
a communication device configured to transmit the encrypted first bio-signal to an external device.

2. The electronic device of claim 1, wherein the first encryption method is performed by applying a constant to the one or more second bio-signals to obtain weighted one or more second bio-signals, and by combining the weighted one or more second bio-signals with the first bio-signal.

3. The electronic device of claim 2, wherein the constant is a predetermined time delay constant.

4. The electronic device of claim 1, wherein the encryption chip is configured to encrypt the one or more second bio-signals by a second encryption method,
wherein the communication device is configured to transmit the encrypted one or more second bio-signals along with the first bio-signal.

5. The electronic device of claim 1, wherein the plurality of bio-signals are signals that change continuously over time, and comprise at least one of an electrocardiogram signal, blood pressure, blood glucose, heart rate, oxygen saturation (SpO₂), arterial stiffness, stress index, body fat, and body temperature.

6. The electronic device of claim 1, further comprising a display configured to display the plurality of bio-signals before encryption is applied to the plurality of bio-signals.

7. The electronic device of claim 6, wherein the display comprises a user interface configured to receive a user input that indicates the first bio-signal as an encryption target.

8. The electronic device of claim 1, further comprising a decryption chip configured to receive an encrypted third bio-signal and unencrypted one or more fourth bio-signals, decrypt the encrypted third bio-signal by using the unencrypted one or more fourth bio-signals as a decryption code, and the decrypted third bio-signal.

9. The electronic device of claim 1, wherein the encryption chip is configured to generate a decryption key based on the one or more second bio-signals, and the communication device is further configured to share the decryption key with the external device.

10. The electronic device of claim 1, wherein the first bio-signal and the one or more second bio-signals are different types of bio-signals which are simultaneously measured by the sensor.

11. A bio-signal encryption chip comprising:
a receiver configured to receive a first bio-signal and one or more second bio-signals;
a converter configured to convert the first bio-signal and the one or more second bio-signals into digital signals; and
a processor configured to encrypt the converted first bio-signal based on a predetermined encryption method that uses the converted one or more second bio-signals as an encryption code, and output the encrypted first bio-signal.

12. The bio-signal encryption chip of claim 11, wherein the predetermined encryption method is performed by applying a constant to the converted one or more second bio-signals to obtain weighted one or more second bio-signals, and by combining the weighted one or more second bio-signals with the converted first bio-signal.

13. The bio-signal encryption chip of claim 12, wherein the constant is a predetermined time delay constant.

14. A method of encrypting bio-signals by an electronic device, the method comprising:
measuring a plurality of bio-signals that comprises a first bio-signal and one or more second bio-signals;
encrypting the first bio-signal based on a first encryption method that uses the one or more second bio-signals as an encryption code; and
transmitting the encrypted first bio-signal to an external device.

15. The method of claim 14, wherein the first encryption method is performed by applying a constant to the one or more second bio-signals to obtain weighted one or more second bio-signals and by combining the weighted one or more second bio-signals with the first bio-signal.

16. The method of claim 15, wherein the constant is a predetermined time delay constant.

17. The method of claim 14, further comprising encrypting the one or more second bio-signals by a second encryption method, and transmitting the encrypted one or more second bio-signals along with the first bio-signal to the external device.

18. The method of claim 14, further comprising displaying the plurality of bio-signals before encryption is applied to the plurality of bio-signals.

19. The method of claim 14, further comprising:
generating a decryption key based on the one or more second bio-signals; and
sharing the decryption key with the external device.
